(19) United States
(12) Patent Application Publication
(10) Pub. No.: US 2026/0250076 A1
(43) Pub. Date: Aug. 27, 2026

(54) CARRYING APPARATUS

(71) Applicant: FINATEC HOLDING AG, Brügg bei Biel (CH)

(72) Inventors: Andreas LISSER, Zuchwil (CH); Nicolas GAUTSCHI, Aarberg (CH); Peter BURCKHARDT, Leubringen (CH); Patrick BROSSARD, Lyss (CH); Hansjörg KLOCK, Worb (CH); Fridolin MAIBACH, Safnern (CH); Matthias HERMLE, Brügg bei Biel (CH)

(21) Appl. No.: 18/881,984

(22) PCT Filed: Jun. 23, 2023
(86) PCT No.: PCT/EP2023/067089

§ 371 Date: Jan. 7, 2025

(30) Foreign Application Priority Data
Sep. 9, 2022 (DE) 10 2022 123 016.3

(51) Int. Cl. B65G 35/06; G01N 33/00

(52) U.S. Cl. CPC B65G 35/06; G01N 33/0078

(57) ABSTRACT

A carrying apparatus (1) of an inspection unit for inspecting parameters of the parts which are produced by in injection molding, punching, deep-drawing or thermoforming machine and are transported through the inspection unit, having a carrying base element (2), having at least two carrying profiles (3), which are oriented parallel to one another, having at least two carrying ribs (4), which connect at least two carrying profiles (3) to one another, are arranged perpendicularly to the longitudinal axis of the carrying profiles (3) and are fastened on these carrying profiles, and also having at least two carrying plates (5), which are fastened on the at least two carrying ribs (4), wherein each carrying plate (5) is arranged at the ends (6) of at least two carrying ribs (4), and is assigned to one of the at least two carrying profiles (3), wherein the carrying apparatus (1) also has a transporting means (7) for the parts, the transporting means being fastened on at least two of the carrying plates (5).

The invention pertains to a carrying apparatus of an inspection unit for the inspection, particularly the inline or resorting inspection, of parameters of parts that are produced by an injection molding, punching, deep-drawing or thermoforming machine and transported through the inspection unit.

A number of quality controls are carried out in the course of the production of injection-molded parts—such as beverage caps—or other plastic or metal parts by means of injection molding, punching, deep-drawing or thermoforming, wherein each individual part is inspected, among other things, with respect to completeness and freedom from defects. In this context, the quality control of each individual part particularly comprises an optical quality control for completeness and freedom from defects, but also a quality control with respect to color defects, underspray, holes, burrs, gate marks, etc. The parts that are defective in one or more respects are subsequently rejected.

The number and the type of control devices at different points of the production path of the parts significantly influence the space requirement of the production device and especially the production speed. The control processes therefore need to be configured in such a way that they preferably do not slow down the production process, wherein normal parts-related processing speeds, particularly of injection-molded parts, currently amount to more than 2000 parts/min and as much is 4000 parts/min such that less than 30 ms are available per part for a quality control at one location.

In addition to the required speed of the quality controls, they must also be as precise as possible in order to prevent an unnecessarily large number of rejects, wherein imprecise quality controls can even lead to defective parts not being detected as such and therefore not being rejected. However, the precision typically is neglected in favor of focusing the function of the inspection unit on the high number of parts per minute. In the prior art, for example, it is common practice to hold in position a transport device such as a vacuum conveyor belt, by means of which the parts are transported through the inspection unit, by means of sheet metal or aluminum profiles with the aid of a screw connection with a through hole. These sheet metal profiles are also used for holding in position inspection units such as sensors, which in most cases are pre-calibrated under laboratory conditions. Due to this simple fastening, the transport device can be very easily removed from the device for the regularly required belt change. However, the production-related tolerances of the sheet metal profiles and the fastening holes, as well as the inspection units that cannot be readily calibrated under operating conditions, lead to sacrifices regarding the precision of the quality control. Even if the inspection units are calibrated to the new conveyor belt being used, this additional work step always has to be carried out anew during each further belt change or other maintenance work that leads to mechanical deviations from the previous state.

The present invention therefore is based on the objective of proposing a carrying apparatus of an inspection unit, which is particularly precise despite the high requirements regarding the throughput rate of an inline or resorting inspection device, and thereby solving the aforementioned problems of the prior art.

This objective is attained by means of a carrying apparatus of an inspection unit for the inspection of parameters of parts that are produced by an injection molding, punching, deep-drawing or thermoforming machine and transported through the inspection unit, wherein said carrying apparatus comprises a carrying base element that has at least two carrying profiles that are oriented parallel to one another, at least two carrying ribs that connect the at least two carrying profiles to one another, are arranged perpendicular to a longitudinal axis of the carrying profiles and fastened on these carrying profiles, as well as at least two carrying plates that are fastened on the at least two carrying ribs, wherein each carrying plate is arranged on the ends of at least two carrying ribs assigned to one of the at least two carrying profiles, and wherein the carrying apparatus furthermore has a transporting means for the parts, which is fastened on at least two of the carrying plates.

The following description primarily concerns injection-molded parts produced in an injection molding machine, but, according to the invention, also includes other plastic or metal parts produced by means of injection molding, punching, deep-drawing or thermoforming.

The carrying base element represents the base frame that produces the connection between a substructure at the site of operation and the inspection unit with its inspection elements and, according to the invention, compensates inaccuracies in the substructure such that the inspection elements can be inserted into the inventive carrying base element in a largely standardized and pre-calibrated manner and directly used without further calibrations.

According to the invention, it is particularly important to keep the chain of errors of the carrying base element at a minimum because dimensional errors, e.g. of the carrying rib, can affect the arrangement of the carrying profiles and the carrying plates connected thereto, as well as the transporting means that is directly or indirectly fastened on the carrying plates and the inspection elements. The carrying plates, the transporting means, the inspection elements and their respective connecting elements represent additional sources of dimensional errors, the errors of which add up. The inspection unit and therefore the quality of the control results of this inspection unit stand at the end of this chain of errors.

In addition to this error reduction, the invention ensures the repeatability or repeat accuracy of the measuring system. The term repeat accuracy refers to the precision of the system, due to which the individual parts are also positioned at the same location once again when they are removed and reinstalled, e.g. due to maintenance work that may also include the exchange of components, wherein this advantageously eliminates the need for a recalibration or only makes a recalibration necessary in exceptional situations.

According to the invention, the carrying profiles are arranged parallel to one another, wherein this is ensured in a highly precise manner by the carrying ribs that are respectively connected to the two carrying profiles and oriented perpendicular to the carrying profiles. Two or more carrying profiles are particularly advantageous for protecting the carrying base element against torsion and twisting of the carrying ribs. The inventive carrying ribs as such are highly precise components, particularly milled components, which have the smallest dimensional tolerance possible due to the type of their manufacture. According to the invention, the carrying ribs do not have to be realized in one piece, but may also be composed of multiple pieces because this allows the manufacture of particularly stable and dimensionally accurate carrying ribs. Furthermore, the term “components” refers to all parts of the apparatus that jointly form the inventive carrying apparatus. All recesses, bores or similar features of these components likewise have a correspondingly small error or are manufactured with small tolerances such that a high overall accuracy of the apparatus and therefore of the inspection unit connected thereto is realized.

Analogous to this measure, the carrying plates fastened on the carrying ribs, as well as their dimensions, recesses and bores, also are produced, particularly milled, with a small tolerance. This production with small tolerances particularly is inventive in the region of the connections between the components.

The transporting means that serves for the transport of the parts, particularly injection-molded parts, through the inspection unit is arranged between the two carrying plates and directly or indirectly connected to these carrying plates. The transporting means preferably is a vacuum conveyor belt, but may also be any other transporting means such as a simple conveyor belt. In any case, this transporting means is, according to the invention, likewise connected to the carrying plates with small tolerances such that it is repeatedly arranged within the carrying apparatus in an accurately positioned manner during an exchange. It is likewise advantageous that the surface of the transporting means, on which the parts, particularly injection-molded parts, are moved, is arranged above the surface of the carrying plates, i.e. that the surfaces of the carrying plates are lowered in relation to the surface of the transporting means, such that objects located on the carrying plates cannot readily reach the transporting means and be incorporated into the flow of parts to be inspected.

An embodiment of the invention proposes a modular expansion of the carrying base element, particularly coaxially on both sides of the carrying base element. In this way, an inlet and/or outlet belt of the inventive apparatus can be advantageously extended on site depending on the respective requirements. In order to realize this modular expansion, the length of the carrying profiles and accordingly the number of carrying ribs can be adapted as needed, wherein the basic construction of the carrying base element remains the same. It is likewise possible to subsequently expand the carrying base element, particularly by adding further carrying plates, carrying ribs and/or adapter plates that connect the carrying plates and/or the carrying ribs to one another. In order to ensure the stability of the expanded carrier base element, it is advantageous to produce a screw connection or pin connection of the carrying ribs in the region of the interface of the expansion.

According to an enhancement of the invention, it is proposed that the carrying ribs are fastened, particularly screwed, on the carrying profiles in two directions that extend perpendicular to one another and to the longitudinal axis of the carrying profiles. This screw connection is a simple yet precise and stable type of connection. The connection of the carrying ribs to the carrying profiles in two axes, namely the two axes that are oriented perpendicular to the longitudinal axis of the carrying profile, advantageously increases the stability and the precision of the apparatus. This double connection likewise stabilizes the orientation and the position of the carrying profiles, which thusly orient themselves on the carrying ribs, such that aluminum can also be used as material for the carrying profiles.

An embodiment of the invention proposes that locating pins are arranged between the carrying plates and the carrying ribs and/or between the carrying plates and the transporting means. The locating pins serve for exactly aligning the connection between the components parallel to the respective connecting surface. In this way, a relative displacement between the components to be connected is prevented in the plane extending perpendicular to the longitudinal axis of the locating pins—i.e. parallel to the connecting surface—whereas a displacement parallel to the longitudinal axis of the locating pins is prevented by the connecting elements, e.g. the screws. The locating pins, as well as the recesses for receiving the locating pins in carrying plates and carrying ribs or transporting means, are produced in a particularly exact manner as described above and realized, in particular, in the form of milled parts. As an alternative to the arrangement of locating pins between the carrying plates and the transporting means, it is also possible, according to the invention, to realize centering by means of countersunk screws and, if applicable, oblong holes, which likewise make it possible to align the transporting means, particularly parallel to the longitudinal axes of the carrying profiles.

The carrying plates preferably are arranged relative to one another in a plane that extends parallel to a plane defined by the carrying profiles, wherein the parallelism of the carrying plates to one another is made possible by a parallel design of the respective contact surfaces with the carrying ribs. An exactly identical height, as well as the most precise parallelism possible, is thereby achieved for the carrying plates lying opposite of one another due to their exact design. According to the invention, it is also possible to arrange the carrying plates lying opposite of one another in a vertically offset manner, but at least one of the carrying plates is in this case connected to the transporting means in such a way that the surface of the transporting means, on which the parts, particularly injection-molded parts, are transported, is aligned parallel to the surfaces of the carrying plates. However, the parallel arrangement of the carrying plates in a common plane is preferred over such a design. The transporting means basically is connected to the carrying base element, particularly the carrying plates, in such a way that the transporting means is arranged parallel to the carrying plates and parallel to the plane defined by the carrying profiles. This ensures that the orientation of the inspection elements connected to the carrying plates is adapted to the orientation of the carrying plates in order to simultaneously also be calibrated to the orientation of the parts, particularly injection-molded parts, on the transporting means.

An embodiment of the invention proposes that the carrying plates and the transporting means have fastening elements that correspond to one another and particularly are arranged in or extend through projections that respectively protrude from the carrying plate and from the transporting means. The fastening elements particularly are screws and locating pins. The corresponding projections and their surfaces that contact one another in the connected state are exactly parallel to one another and, according to the invention, may likewise be arranged parallel to the carrying plates and the transporting means, particularly in consideration of the available space. The direct connection of the carrying plates and the transporting means furthermore minimizes the number of sources of error in the system.

According to an enhancement of the invention, it is proposed that it comprises a pivoting frame that is positioned above the transporting means and connected to the carrying plates, particularly by means of locating pins and screws, wherein the pivoting frame can be pivoted in such a way that the inspection elements arranged thereon and the transporting means lying thereunder are accessible.

This pivoting frame allows a modular installation of the inspection elements. Due to its pivotability, the respective inspection units/elements arranged thereon and the transporting means lying thereunder are accessible. In this case, it is also essential that the working position of the pivoting frame or, in other words, its non-pivoted end position is a repeatable position, i.e. that the pivoting frame can always be moved into exactly the same position.

The pivoting frame has a pivotably mounted part that is fastened on the carrying plates on its end—having the pivoting axis. The other, freely pivotable end particularly rests on stops in a closed position of the pivoting frame, wherein said stops are in turn fastened on the carrying plates. To this end, it is particularly advantageous that the pivoting frame has corresponding spring-loaded indexing bolts, which in the closed position engage into corresponding latching openings in the stops and thereby lock the pivoting frame in this exact closed position. The lock can be disengaged, for example, with the aid of a pushbutton on the free end of the pivoting frame. The stops have lead-in bevels in order to enable the indexing bolts to automatically slide into the latching holes when the pivoting frame is closed.

The pivoting frame particularly is exactly fastened on the carrying plates with the aid of locating pins and screws. The same applies to the stops. The enhancement of the invention with inscribed scales in each adjustable position makes a repositioning process reproducible and repeatable.

The pivotable part of the pivoting frame has a base frame for mounting any inspection elements required, wherein said base frame particularly has positioning scales, which are formed by multiple recesses that preferably are inscribed. The positioning scales thereby allow a repeatable installation of inspection elements. The inspection elements can be modularly inserted into this base frame at will. To this end, the inspection elements are installed in a pivoting frame module, which in addition to the inspection element has positioning plates that can be separably connected to the base frame of the pivoting frame. A particularly exact design of the base frame and the positioning plates is also essential to the invention in this case and a connection to one another by means of locating pins and screws is particularly advantageous. The positioning plates and the base frame preferably are provided with a scale. The pivoting frame modules allow the use of inspection elements that can be modularly inserted into the base frame of the pivoting frame in the form of preassembled units and are exchangeable. The base frame accommodates one or more pivoting frame modules depending on the respective requirements. As an alternative to inspection elements, a pivoting frame may also accommodate another transporting means such as a vacuum conveyor belt in order to transport the parts to be inspected over its upper side such that its underside is freely accessible for inspection purposes.

An embodiment of the invention proposes that the pivoting frame has at least one pivoting force booster, particularly a pneumatic spring, and/or positioning radii that correspond to one another, wherein the positioning radii are arranged in at least one stop for the pivoting frame and in a pivotable free end of the pivoting frame. A pivoting force booster simplifies the movement of the pivoting frame, which is rather heavy when fully loaded, for a user without requiring particular exertion. This also significantly reduces the risk of damaging the exact elements of the pivoting frame as a result of an excessively brisk operation, wherein this particularly applies to a damping of the closing movement of the pivoting frame. The positioning radius in the stop and the corresponding positioning radius in the free end of the pivoting frame preferably are milled parts and therefore components in the sense of the invention as described above.

According to an enhancement of the invention, it is proposed that the carrying base element has at least one inspection element that is selected from the following group: a camera, a lens, a lighting assembly, a sensor for tracking parts—particularly a trigger light barrier—, a sensor for counting parts—particularly a counting light barrier—, a conveyor belt, a blow-off valve, a discharge chute, a separating device, an extraneous light protection, a high-voltage testing unit and a parts measuring unit. In this context, the inspection elements comprise any elements that are conceivable or required for the quality control of the parts, particularly injection-molded parts.

An embodiment of the invention proposes that the carrying ribs, the carrying plates and/or the fastening elements or parts thereof are realized in the form of milled parts. If only parts of the carrying ribs, carrying plates and/or fastening elements are realized in the form of milled parts, this particularly concerns their connecting parts for producing a connection with other components. This measure primarily minimizes the total error.

According to an enhancement of the invention, it is proposed that the carrying base element is indirectly connected to a stationary substructure, particularly a work table or a work cabinet, by means of connecting elements. In this context, the work table or the work cabinet as such does not necessarily form part of the invention. In fact, it is possible to choose any other substructure that can respectively accommodate or carry the carrying base element and is present or should be used at the installation site. The dimensions of the substructure do not have to be particularly exact due to this enhancement of the invention. The particularly exact carrying base element is not affected by the error-prone substructure because the connecting elements compensate these errors. To this end, the connecting element has oblong holes.

According to the invention, the work table or the work bench (commonly referred to as “work table” below) not only comprises a base frame, but particularly also two or more sidewalls. In addition, horizontally arranged transverse sheet metals and vertically arranged center walls are advantageous for increasing the stability of the work table. According to the invention, it is advantageous to connect the carrying base element to the center walls of the work table directly or by means of the above-described connecting elements, e.g. screws.

The carrying base element is directly screwed on the center walls with one respective fastening rib. It intentionally does not rest on the sidewalls so as to not transfer the inaccuracy of the work table to the carrying base element. The connection to the sidewalls is produced by means of oblong holes in the connecting piece, wherein a clearance zone between the respective sidewall and the carrying base element is always preserved. Since the carrying base element only is connected to the two closely adjacent center walls in a non-compensating manner, it cannot bend at the variable fastening on the remaining sidewalls. In this case, a slight inclination of the carrying base element relative to a substructure is harmless for the exactness and repeatability of the quality controls because the transporting means and the inspection units are arranged at the same angle to the substructure and therefore not inclined relative to one another due to the exact carrying base element. Consequently, the angular error does not affect the performance of the quality control.

According to yet another enhancement, it is proposed that the work table accommodates an electrical cabinet and/or a fan and/or a reject container. These elements are required for the operation of the inspection units and the transporting means and for accommodating the rejected parts and are made available at the site of operation.

A hood on the carrying plates or on the work table protects the inspection area from external influences. This hood can be separably connected and/or at least partially pivoted such that the region lying thereunder is accessible for maintenance work. The hood may have doors or removable walls.

In other respects, it is advantageous if preferably all mechanical elements, which can be adjusted for calibrating the inspection, have a scale and/or mechanical guides that, according to the invention, are once again designed as exact as possible. Due to these scales and the mechanical guides, errors in the calibration are prevented and repeatability in each variable element of the apparatus is ensured.

As an example, a preferred embodiment of the invention is described below with reference to the drawings, wherein further advantageous details can be gathered from said drawings.

Functionally identical parts are identified by the same reference symbols in all drawings.

In these drawings:

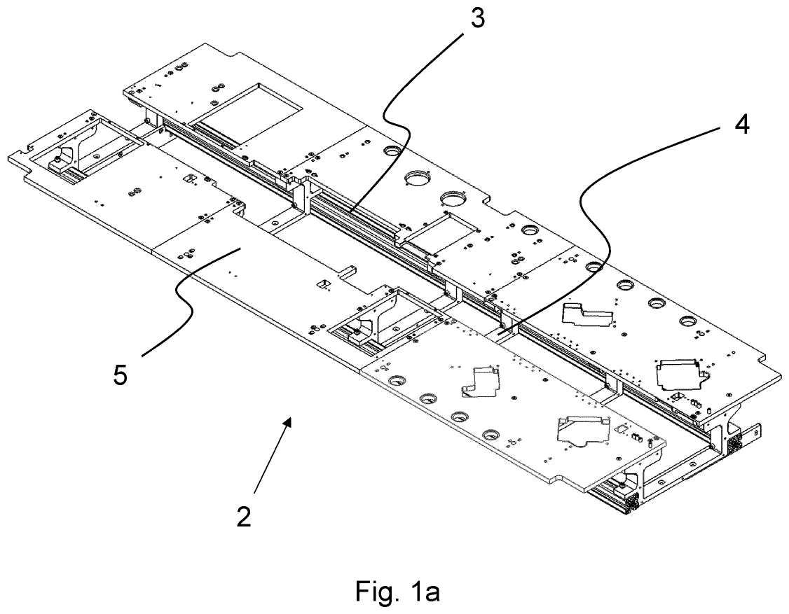

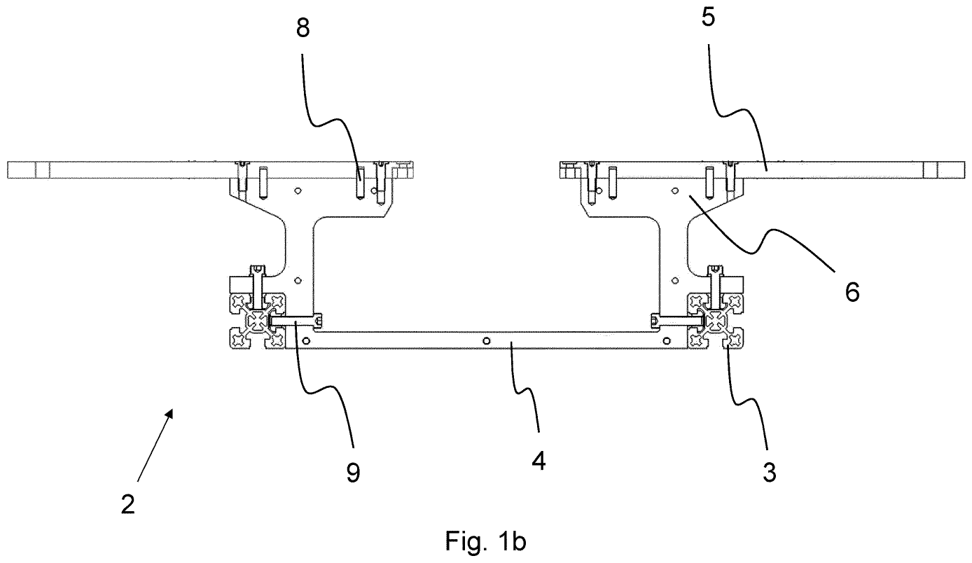

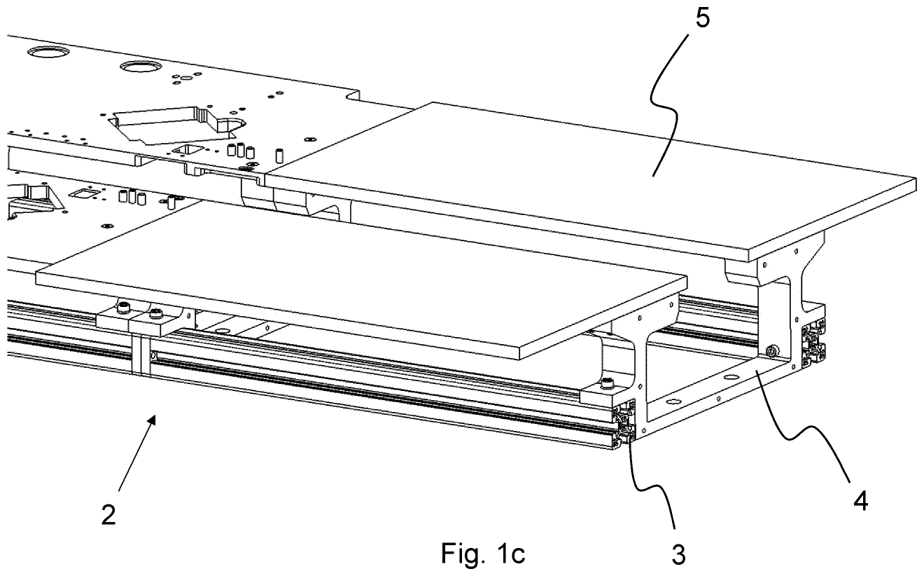

FIG. 1a shows a schematic view of an inventive carrying base element,

FIG. 1b shows a sectional view of an inventive carrying base element,

FIG. 1c shows a schematic view of an inventive carrying base element with a modular extension,

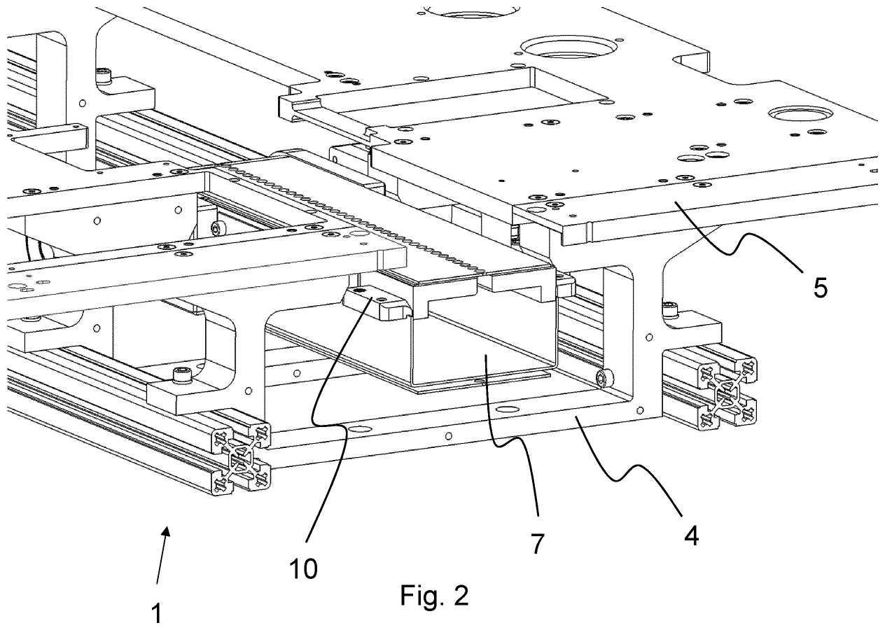

FIG. 2 shows a schematic view of an inventive carrying base element with transporting means in the disconnected state,

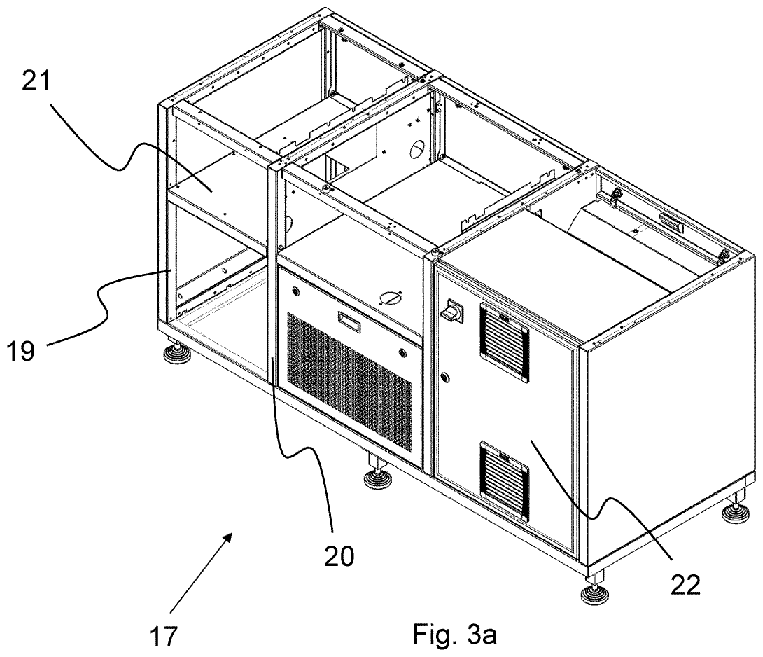

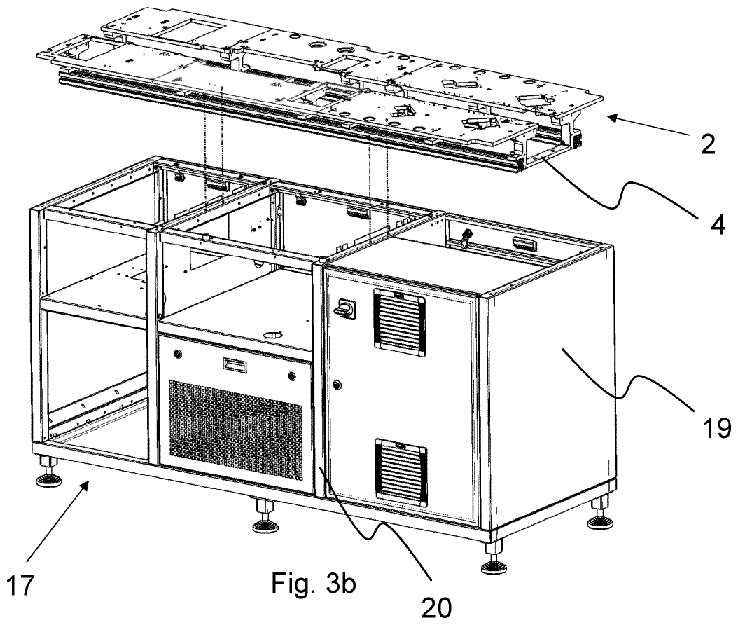

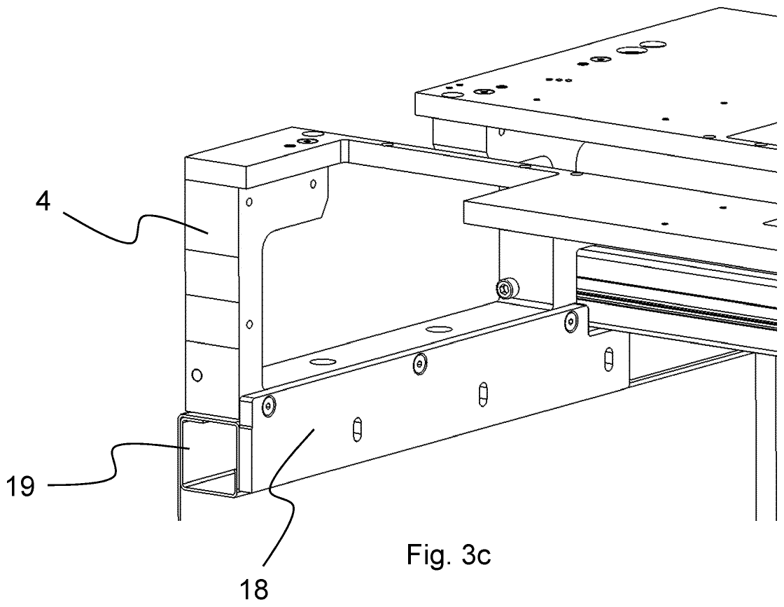

FIG. 3a shows a schematic view of an inventive work table,

FIG. 3b shows a schematic view of an inventive work table with an inventive carrying base element in the disconnected state,

FIG. 3c shows a schematic view of an inventive work table with an inventive carrying base element in the connected state,

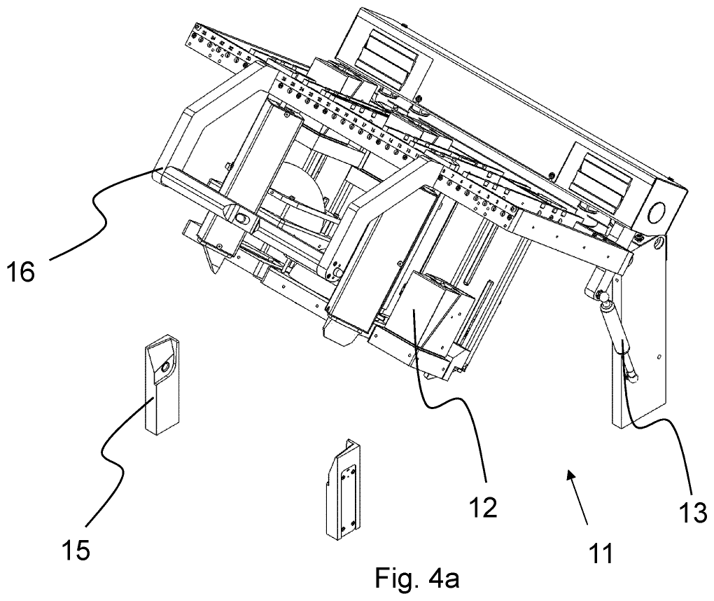

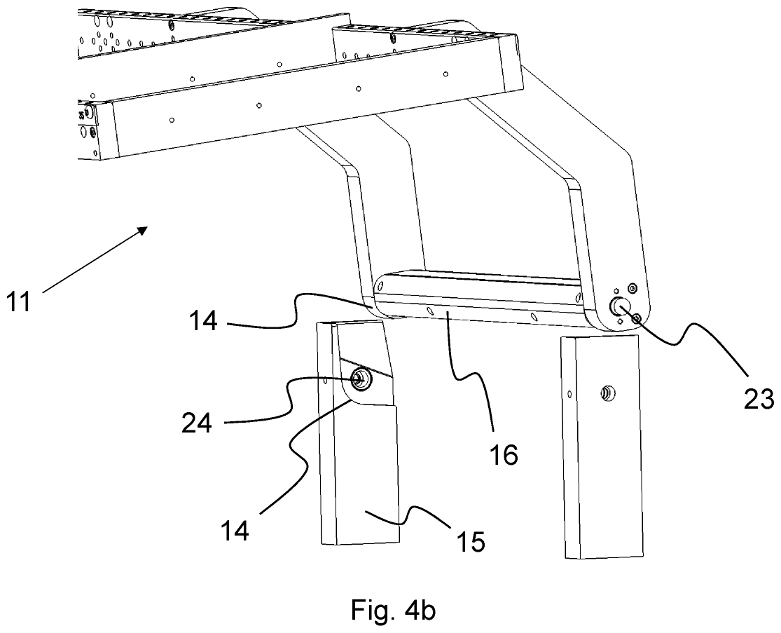

FIG. 4a shows a schematic view of an inventive pivoting frame in the opened state,

FIG. 4b shows a schematic view of a closing mechanism of an inventive pivoting frame,

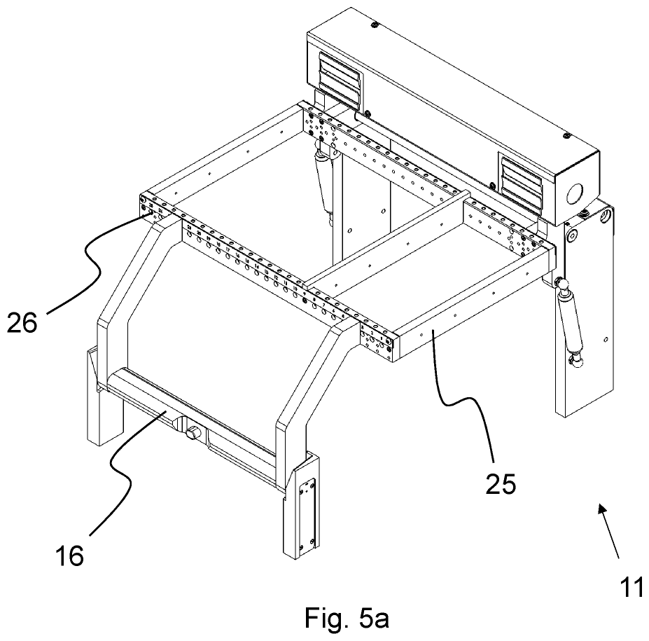

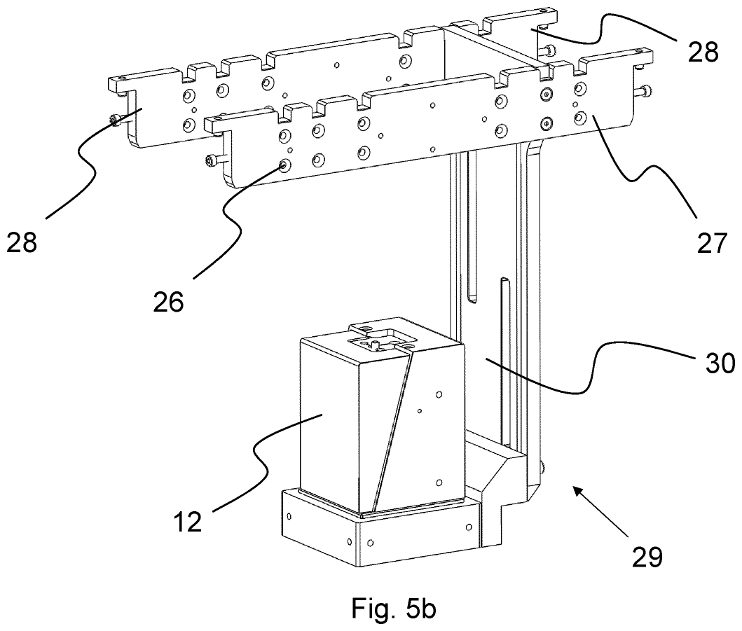

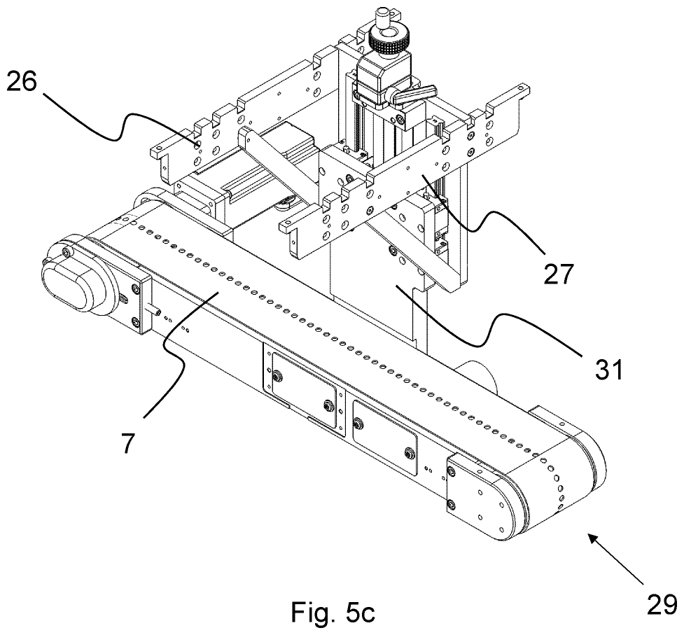

FIG. 5a shows a schematic view of an inventive pivoting frame in the closed state without installed pivoting frame modules,

FIG. 5b shows a schematic view of a first inventive pivoting frame module, and

FIG. 5c shows a schematic view of a second inventive pivoting frame module.

FIG. 1a shows a schematic view of an inventive carrying base element 2 of the carrying apparatus 1. The carrying base element 2 is composed of two parallel carrying profiles 3 and multiple carrying ribs 4, as well as multiple base plates 5. The carrying ribs 4 are respectively arranged parallel to one another and perpendicular to the carrying profiles 3, wherein the carrying ribs are connected to the carrying profiles, particularly in the angled region of the carrying ribs 4 shown, such that all carrying ribs 4 have the same orientation. The carrying profiles 3 are conventional aluminum profiles whereas the carrying ribs 4 and the carrying plates 5 are respectively realized or partially realized in the form of milled parts. The carrying plates 5 are arranged on the two free ends of the carrying ribs 4 and arranged relative to one another in such a way that the carrying plates 5 can be respectively assigned to one end of the carrying ribs 4 or to one carrying profile 3. In this case, the carrying plates 5 assigned to the one carrying profile 3 lie opposite of the carrying plates 5 assigned to the other carrying profile 3. In addition, the carrying plates 5 lying opposite of one another are spaced apart from one another such that a clearance zone is formed between the opposing carrying plates 5. The carrying base element illustrated in this figure comprises exactly six carrying plates 5 and six carrying ribs 4, but the carrying base element may, according to the invention, comprise any number of carrying plates 5 and carrying ribs 4 that is greater than or equal to two. The two upper carrying plates 5 in the figure are connected to two carrying ribs 4 whereas the two lower carrying plates 5 in figure are connected to three carrying ribs 4. The two central carrying ribs 5 and the two upper carrying plates 5 share a carrying rib 4, on which four carrying plates 5 are collectively fastened. All carrying plates 5 are always connected to at least two carrying ribs in order to ensure a sufficient stability of the carrying plates 5. The recesses in the carrying plates 5 serve for receiving different inspection units 12, which are not illustrated in this figure, whereas the smaller bores shown serve for receiving fastening elements 9 or locating pins 8 of any inspection units 12 arranged on the carrying plates 5.

FIG. 1b shows a sectional view of an inventive carrying base element 2. The carrying profiles 3 and the carrying ribs 4 are jointly fastened in the region of the two angles of the carrying ribs 4 by means of fastening elements 9. The carrying ribs 4 are screwed to the carrying profiles 3 along two axes that extend perpendicular to one another and to the longitudinal axis of the carrying profiles 3, particularly by using sliding blocks. In addition, the carrying ribs 4 and the carrying plates 5 are realized in the form of milled parts that have particularly small fault tolerances. The carrying plates 5, which are respectively connected to the free ends 6 of the carrying ribs 4, are aligned exactly parallel to one another. In addition, their position in the parallel plane of the carrying plates 5 is once again ensured with the aid of locating pins 8 and not-shown fastening elements 9. The locating pins 8 and/or the fastening elements 9 are turned parts whereas the corresponding recesses in the carrying plates 5 are milled in order to achieve the smallest error possible in the system.

FIG. 1c shows a schematic view of an inventive carrying base element 2 with a modular extension. The added element comprises two carrying plates 5, two carrying ribs 4 and two carrying profiles 3. The expanded carrying profiles 3 are arranged coaxially to the two original carrying profiles. The expanded carrying ribs 4 and carrying plates 5 are connected to the expanded carrying profiles 3, wherein the fastening elements 9 and locating pins 8 used for producing this connection are identical to those used for the original carrying base element 2. The connection of the original carrying base element 2 to the expanded carrying base element 2 is produced by means of the two carrying ribs 4 that face one another. These carrying ribs are connected to one another, particularly also by means of locating pins 8 and fastening elements 9, in order to also allow a particularly small fault tolerance in this case. According to the invention, it would likewise be possible to arrange adapter plates between the carrying profiles 3, wherein said adapter plates can be connected to one another and/or adapt the length of the carrying profiles 3 to one another.

FIG. 2 shows a schematic view of an inventive carrying base element 2 with transporting means 7 in the disconnected state. In the embodiment shown, the carrying apparatus 7 is directly connected to the carrying plates 5 and not in direct contact with the carrying ribs 4. In order to produce the connection between the transporting means 7 and the carrying plates 5, both parts respectively have projections 10, the mutual contact surfaces of which are milled or otherwise designed in an exact manner. The parallel arrangement of the carrying plates 5 is transferred to the transporting means 7 in such a way that this transporting means is arranged exactly parallel to the two carrying plates 5. Recesses that guide the fastening elements 9 furthermore are provided in the protruding projections 10.

FIG. 3a shows a schematic view of an inventive work table 17. In addition to a base frame, this work table has two sidewalls 19 and two central walls 20. The work table 17 furthermore has horizontally arranged transverse sheet metals 21 in two regions that are delimited by the sidewalls 19 and the central walls 20, wherein said transverse sheet metals, as well as the center walls 20, increase the stability of the work table 17. The electrical cabinet 22 arranged between a center wall 20 and a sidewall 19 likewise improves the stability of the work table 17. The inspection units 12, which are subsequently positioned on the work table 17 by means of the carrying apparatus 1, can be supplied with energy by means of the electrical cabinet 22. In this case, the parts of the work table 17 are not necessarily designed in an exact manner because the dimensional tolerances of the parts are compensated by the exact carrying base element 2 and the connection between the carrying base element 2 and the work table 17.

FIG. 3b shows a schematic view of an inventive work table or work cabinet 17 with an inventive carrying base element 2 in the disconnected state. The work table 17 is at least indirectly connected to the carrying base element 2 arranged above the work table by means of the sidewalls 19 and the center walls 20. In the embodiment shown, the carrying base element 2 is directly connected, particularly directly screwed, to the two center walls 20 of the work table 17 by means of its carrying ribs 4, wherein the outer carrying ribs 4 of the carrying base element 2 only are indirectly connected by means of connecting elements 18. Since the carrying base element 2 is directly connected to exactly two center walls 20, a distortion or deformation of the carrying base element 2 is precluded due to different heights of the center walls 20 and only a uniform inclination of the entire carrying base element 2 is possible. This inclination is irrelevant because all devices fastened on the carrying base element 2 have the same inclination relative to the substructure and therefore are not inclined relative to one another.

FIG. 3c shows a schematic view of an inventive work table 17 with an inventive carrying base element 2 in the connected state. This figure shows a close-up view of the connection between the sidewall 19 of the work table 17 and the carrying rib 4 of the carrying base element 2 as illustrated in FIG. 3b. In this case, the connection between the sidewall 19 and the carrying rib 4 is produced indirectly by means of connecting element 18. The connecting element 18 respectively has recesses for receiving fastening elements, in this case screws, for the carrying rib 4 and for the sidewall 19. In this context, only the recesses that are assigned to one of the two parts to be connected, in this case to the carrying rib 4, are not realized in the form of oblong holes. In contrast, the recesses assigned to the sidewall 19 are oblong holes in order to allow a position correction. The carrying rib 4 and the sidewall 19 do not have to contact one another, i.e. a gap between these two parts is permissible.

FIG. 4a shows a schematic view of an inventive pivoting frame 11 in the opened state. The pivoting frame 11 shown essentially is composed of a pivotable part and two stops 15. The stops 15 form an exact delimitation of the pivoting movement of the pivotable part of the pivoting frame 11. The pivotable part has a free end 16, which respectively rests on or is received by the stops 15 in a closed state of the pivoting frame 11. The pivoting frame 11 serves for accommodating one or more inspection elements 12, which due to the pivotability of the pivoting frame 11 can be moved between an operating state and an accessible state, e.g. for maintenance work or the exchange of the inspection elements 12. Pneumatic springs 13 simplify opening and closing of the pivoting frame 11 and therefore protect the stops 15 against damages.

FIG. 4b shows a schematic view of a closing mechanism of an inventive pivoting frame 11. This figure shows the two stops 15 and the freely pivotable end 16 of the pivoting frame 11, which respectively rests on or is received by the stops 15 in a closed position. The surfaces that contact one another in the closed state—in this case positioning radii 14 that correspond to one another—are components in the sense of the invention and therefore manufactured, particularly milled, with minimal tolerances. The free end 16 of the pivoting frame 11 has lateral indexing bolts 23, which in the closed state of the pivoting frame 11 engage into the latching openings 24 in the stops 15 such that the pivoting frame 11 is locked in the closed position. A pushbutton illustrated, for example, in FIG. 5a is provided on the free end 16 of the pivoting frame 11 in order to disengage this lock, wherein a user can retract the spring-loaded indexing bolts 23 and thereby pull the indexing bolts out of the latching openings 24 by actuating said pushbutton. The stops 15 have beveled guide planes (lead-in bevels) in order to enable the indexing bolts 23 to easily and automatically slide into the latching openings 24 when the pivoting frame is closed, wherein these beveled guide planes gradually push in the indexing bolts 23 when the pivoting frame 11 is closed until they are positioned above the latching openings 24 and reset therein.

FIG. 5a shows a schematic view of an inventive pivoting frame 11 in the closed state without installed pivoting frame modules 29. The basic structure of the pivoting frame 11 therefore primarily consists of a base frame 25 that is mounted in the pivotable part of the pivoting frame 11. All required inspection elements 12 can be attached to this base frame 25 in a customized and modular manner. In addition, the inspection elements 12 can be exchanged at any time as needed. This exchange can also be carried out in a repeatable manner and without calibrations. This is achieved, in particular, due to the exact design of the base frame 25 with the smallest fault tolerances possible. In addition, the base frame 25 has a positioning scale 26, by means of which the inspection elements 12 or the pivoting frame modules 29 can always be attached to the base frame 25 in the same position without requiring a complex documentation of the breadboard model. In order to ensure repeatability of the inspection set-up, the pivoting frame modules 29 accordingly are primarily limited to the connection to the base frame 25 by means of this positioning scale 26 and its recesses. Consequently, the recesses of the positioning scale 26 also have the smallest fault tolerance possible, wherein this can be achieved, for example, by realizing the base frame 25 in the form of a milled part.

FIG. 5b shows a schematic view of first inventive pivoting frame module 29. The pivoting frame module 29 shown is composed of an inspection element 12, in this example a camera, and a bracket, by means of which the pivoting frame module 29 can be attached to the base frame 25 of the pivoting frame 11. To this end, the pivoting frame module 29 that has two positioning plates 27 that are attached to the positioning scale 26 of the base frame 25 of the pivoting frame 11 with their two ends 28. The positioning plates 27 are also designed in a particularly exact manner in order to produce the connection between the pivoting frame module 29 and the base frame 25 of the pivoting frame 11 as precisely and repeatably as possible. In addition, the positioning plates 27 have on their ends 28 a fastening device, which is oriented along the longitudinal axis of the positioning plates 27, as well as perpendicular thereto, such that the most exact connection possible to the base frame 25 can be produced. Each of the positioning plates 27 likewise has a positioning scale 26. In this example, the inspection element 12 is indirectly connected to the positioning plates 27 by means of a rail 30. The rail 30 not only makes it possible to mount the inspection elements 12 exactly, but also adjustably, wherein said inspection elements are mechanically guided in a vertically adjustable manner due to the rail 30.

FIG. 5c shows a schematic view of a second inventive pivoting frame module 29. This pivoting frame module also has the above-described positioning plates 27 for producing a repeatable connection to the base frame 25 of the pivoting frame 11. However, the pivoting frame module 29 according to this exemplary embodiment carries a transporting means 7 that may transport the parts, particularly injection-molded parts, from above, e.g. for the background inspection of said parts. Depending on the size of the parts, particularly inspection-molded parts, to be inspected, it is sensible to also mount this transporting means 7 in a vertically adjustable manner. To this end, a vertically displaceable adjusting unit 31 is provided. In order to additionally ensure the repeatability of the arrangement, the adjusting unit 31 is not only freely displaceable, but also connected to the positioning scale 26 of the positioning plates 27 by means of cross braces. The positioning plates 27, as well as the adjusting unit 31, are once again designed with the smallest fault tolerance possible.

### List of Reference Symbols

1 Carrying apparatus
2 Carrying base element
3 Carrying profile
4 Carrying rib
5 Carrying plate
6 End of a carrying rib
7 Transporting means
8 Locating pin
9 Fastening element
10 Protruding projection
11 Pivoting frame
12 Inspection element
13 Pneumatic spring
14 Positioning radius
15 Stop
16 Free end of pivoting frame
17 Work table/work cabinet
18 Connecting element
19 Sidewall
20 Center wall
21 Transverse sheet metal
22 Electrical cabinet
23 Indexing bolt
24 Latching opening
25 Base frame
26 Positioning scale
27 Positioning plate
28 Positioning plate end
29 Pivoting frame module
30 Rail
31 Adjusting unit

1. A carrying apparatus (1) of an inspection unit for the inspection of parameters of parts that are produced by an injection molding, punching, deep-drawing or thermoforming machine and transported through the inspection unit, said carrying apparatus comprising:
a carrying base element (2) that has at least two carrying profiles (3) that are oriented parallel to one another, at least two carrying ribs (4) that connect the at least two carrying profiles (3) to one another, the carrying ribs being arranged perpendicular to a longitudinal axis of the carrying profiles (3) and fastened on these carrying profiles, as well as at least two carrying plates (5) that are fastened on the at least two carrying ribs (4),
wherein each carrying plate (5) is arranged on the ends (6) of at least two carrying ribs (4) assigned to one of the at least two carrying profiles (3), and
wherein the carrying apparatus (1) furthermore has a transporting means (7) for the parts, which is fastened on at least two of the carrying plates (5).

2. The carrying apparatus (1) according to claim 1, wherein the carrying ribs (4) are fastened on the carrying profiles (3) in two directions that extend perpendicular to one another and to the longitudinal axis of the carrying profiles (3).

3. The carrying apparatus (1) according to claim 1, comprising locating pins (8) arranged between the carrying plates (5) and the carrying ribs (4) and/or between the carrying plates (5) and the transporting means (7).

4. The carrying apparatus (1) according to claim 1, wherein the carrying plates (5) are arranged in a common plane, wherein this plane extends parallel to a plane defined by the carrying profiles (3).

5. The carrying apparatus (1) according to claim 1, wherein the carrying plates (5) and the transporting means (7) have fastening elements (9) that correspond to one another and particularly are arranged in or extend through projections (10) that respectively protrude from the carrying plate (5) and from the transporting means (7).

6. The carrying apparatus (1) according to claim 1, comprising a pivoting frame (11) that is positioned above the transporting means (7) and connected to the carrying plates (5), wherein the pivoting frame (11) can be pivoted in such a way that the inspection elements (12) arranged thereon and the transporting means (7) lying thereunder are accessible.

7. The carrying apparatus (1) according to claim 1, wherein the pivoting frame (11) has at least one pivoting force booster and/or positioning radii (14) that correspond to one another, wherein the positioning radii (14) are arranged in at least one stop (15) for the pivoting frame (11) and in a pivotable free end (16) of the pivoting frame (11).

8. The carrying apparatus (1) according to claim 1, wherein the carrying base element (2) has at least one inspection element (12) that is selected from the following group: a camera, a lens, a lighting assembly, a sensor for tracking parts, a sensor for counting parts, particularly a counting light barrier, a conveyor belt, a blow-off valve, a discharge chute, a separating device, an extraneous light protection, a high-voltage testing unit and a parts measuring unit.

9. The carrying apparatus (1) according to claim 1, wherein the carrying ribs (4), the carrying plates (5) and/or the fastening elements (9) or parts thereof are realized in the form of milled parts.

10. The carrying apparatus (1) according to claim 1, wherein the carrying base element (2) is indirectly connected to a stationary substructure by means of connecting elements (18).
